(19) United States
(12) Patent Application Publication
(10) Pub. No.: US 2009/0217924 A1
(43) Pub. Date: Sep. 3, 2009

(54) Treatment systems and methods

(76) Inventor: Gregory William Pearson, Houston, TX (US)

Correspondence Address: Hickman Patent Law Offices;attn: William Hickman, PO Box 2202, Bellaire, TX, 77401, US

(21) Appl. No.: 12/072,782

(22) Filed: Feb. 28, 2008

(51) Int. Cl. A61M 11/00; A61K 31/70; A61P 17/10

(52) U.S. Cl. 128/200.14; 514/24

(57) ABSTRACT

There is disclosed a system comprising a fluid reservoir, a liquid solution within the reservoir, the solution comprising an antibiotic of the lincomycin family, a spray nozzle, a spray activator, and a tubular connection connecting the spray nozzle and the fluid reservoir.

### FIELD OF THE INVENTION

The present invention relates to systems and methods for applying dermatological treatments.

### DESCRIPTION OF THE RELATED ART

Clindamycin Phosphate is a known antibiotic used topically for the treatment of acne vulgaris. Clindamycin Phosphate acts by stopping the spread of bacteria that cause acne. Clindamycin Phosphate may be administered as a foam, commercially available as Evoclin®; topical solution, lotion, or gel, commercially available as Cleocin T®; or in a gel combined with benzoyl peroxide, commercially available as Duac®.

Currently available versions of Clindamycin Phosphate are difficult to use on large areas of the body such as the chest or back. Foams, lotions, and gels are time consuming to apply to such large areas, and less of the medicine may reach the skin if there is hair in the area. The currently available methods of application of Clindamycin Phosphate may lead to low patient compliance.

U.S. Patent Application Publication Number 2007/0244195 discloses methods of increasing the efficacy of peroxides such as benzoyl peroxide in the treatment of skin conditions such as acne. Such as methods of increasing radicals formed by peroxides on/in the skin, more specifically near/in the comedone, for topical use in dermatology, or the use of transitional metals such as Cu(I) and ferrous ions to increase the efficacy of peroxides such as benzoyl peroxide. There is also disclosed: a method by which a peroxide such as benzoyl peroxide and its activator are added to the skin surface at the same time; the use of a more soluble form of peroxide such as benzoyl peroxide to increase its efficacy; the addition of a side chain to a peroxide such as benzoyl peroxide so that it is activated by light; the addition of a tertiary amine to a peroxide such as benzoyl peroxide at the time of skin application, to improve the efficacy of the peroxide; and the addition of dapsone or other material to a peroxide such as benzoyl peroxide to improve its efficacy. U.S. Patent Application Publication Number 2007/0244195 is herein incorporated by reference in its entirety.

U.S. Patent Application Publication Number 2006/0137684 discloses methods, compositions, kits and treatment regimens for applying active ingredients to the groin area of an individual to treat the symptoms of a vaginal or vulvar condition using a spray applicator. U.S. Patent Application Publication Number 2006/0137684 is herein incorporated by reference in its entirety.

U.S. Pat. No. 5,446,028 discloses a composition and method for the treatment of acne including a peroxide and an antibiotic selected from the lincomycin family of antibiotics. U.S. Pat. No. 5,446,028 is herein incorporated by reference in its entirety.

U.S. Pat. No. 5,466,446 discloses that dermatological conditions may be treated by topically applying to the affected area an effective amount of a combination benzoyl peroxide and clindamycin composition. U.S. Pat. No. 5,466,446 is herein incorporated by reference in its entirety.

There are needs in the art for one or more of the following: apparatus and methods for applying topical Clindamycin Phosphate solutions which are capable of applying the medication to large areas of the body, to hairy areas, and/or which do not suffer from certain disadvantages of the prior art apparatus and methods.

These and other needs in the art will become apparent to those of skill in the art upon review of this specification, including its drawings and claims.

### SUMMARY OF THE INVENTION

One aspect of invention provides a system comprising a fluid reservoir, a liquid solution within the reservoir, the solution comprising an antibiotic of the lincomycin family, a spray nozzle, a spray activator, and a tubular connection connecting the spray nozzle and the fluid reservoir.

Another aspect of invention provides a method for the treatment of one or more areas of acne vulgaris on a chest or a back of a patient comprising activating a spray activator; and spraying a quantity a liquid solution comprising an antibiotic of the lincomycin family on the one or more areas.

These and other aspects of the invention will become apparent to those of skill in the art upon review of this specification, including its drawings and claims.

### BRIEF DESCRIPTION OF THE DRAWINGS

FIG. 1 shows a spray system.

### DETAILED DESCRIPTION OF THE INVENTION

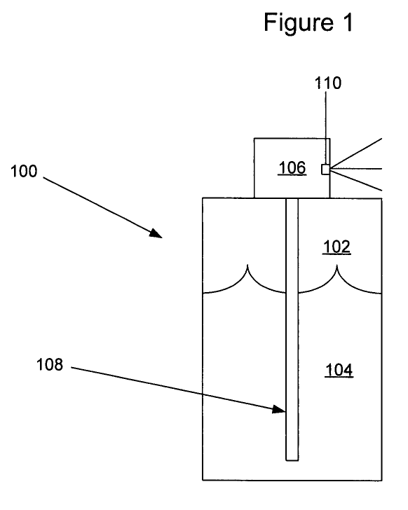

FIG. 1:

Referring now to FIG. 1, there is illustrated system 100. System 100 includes fluid reservoir 102 containing solution 104, spray nozzle 110, spray activator 106, for example an aerosol or manual pump, and a tubular connection 108 from the reservoir to the nozzle. Fluid reservoir may have a capacity from about 20 to about 100 mL, for example from about 30 to about 60 mL.

In operation, a patient may push activator 106 to cause a dosage of the solution 104 to be sucked from fluid reservoir 102 into tubular connection 108 and be forced out of spray nozzle 110. Patient may spray one or more sprays on one or more areas of the back and/or chest that have acne and require treatment.

Each spray may have a volume from about 0.05 mL to about 0.5 mL, for example from about 0.1 mL to about 0.25 mL. One dosage may be from about 1 to about 20 sprays, for example from about 2 to about 10 sprays. One suitable dosage size is from about 0.1 mL to about 5 mL, for example from about 0.5 mL to about 2.5 mL.

One suitable solution includes from about 0.25% to about 50% Clindamycin Phosphate, for example from about 0.5% to about 10% Clindamycin Phosphate, or from about 1% to about 5% Clindamycin Phosphate. One suitable solution also contains isopropyl alcohol 50% v/v, propylene glycol, and purified water.

One suitable solution is a currently available 1% Clindamycin phosphate liquid solution commercially available from Taro Pharmaceuticals USA, Inc. of Hawthorne, N.Y.

Another suitable solution is a currently available Clindamycin liquid solution sold under the tradename Cleocin T commercially available from Pfizer, Inc. of New York, N.Y.

In some embodiments, a Clindamycin liquid solution may also include an effective amount of benzoyl peroxide, for example from about 1% to about 20%, for example from about 2.5% to about 10%, or about 5%.

In some embodiments, an effective amount of a mixture of benzoyl peroxide and clindamycin is any amount which produces the desired results, for example from about 4.5% to about 11% by weight of the total composition of benzoyl peroxide and from about 0.90% to about 2.5% by weight of the total composition of clindamycin. In particular, the ratio of benzoyl peroxide to clindamycin may be from about 1.8:1 to 12:1. Particularly preferred are compositions wherein the ratio of benzoyl peroxide to clindamycin is from about 4:1 to about 5:1. The benzoyl peroxide and clindamycin may be mixed, either separately or together, with one or more inert ingredients which form the pharmaceutical carrier. In a final composition, the benzoyl peroxide may be present in an amount of from about 4.5 to about 11% while the clindamycin may be present in an amount of from about 0.90 to 2.5% of the overall composition. The precise amount of inert ingredients added will depend on the amounts of benzoyl peroxide and clindamycin used to make the final product.

In some embodiments, a suitable pharmaceutical carrier may be employed. Suitable topically acceptable pharmaceutical carriers are those which typically are used in the topical application of pharmaceuticals and cosmetics.

In some embodiments, there may be included a surface active agent or dispersing agent to disperse uniformly the active ingredients. A preferred composition includes a second surface active agent. Such agents include the esters of polyols and sugars, the products of the condensation of ethylene oxide with fatty acids, fatty alcohols, long-chain alkylphenols, long-chain mercaptans, long chain amides, polyethers of polyhydroxylated fatty alcohols and alkylpolyglycol ethers which may be included in an amount of from about 2% to about 6% by weight.

The patient may apply the spray treatment to the affected area one to two times daily for a period of 4 to 8 weeks.

### Illustrative Embodiments

In one embodiment, there is disclosed a system comprising a fluid reservoir, a liquid solution within the reservoir, the solution comprising an antibiotic of the lincomycin family, a spray nozzle, a spray activator, and a tubular connection connecting the spray nozzle and the fluid reservoir. In some embodiments, a quantity of the liquid solution is adapted to be transported from the fluid reservoir to the spray nozzle when the spray activator is engaged. In some embodiments, the quantity comprises from about 0.05 to about 0.5 milliliters for each activation of the spray activator. In some embodiments, the antibiotic comprises clindamycin phosphate or a pharmaceutically acceptable salt or ester thereof. In some embodiments, the liquid solution further comprises a peroxide. In some embodiments, the peroxide comprises benzoyl peroxide. In some embodiments, the liquid solution has a viscosity from about 0.01 to about 100 centipoise. In some embodiments, the spray activator comprises a manual pump. In some embodiments, the liquid solution has a viscosity from about 0.1 to about 10 centipoise. In some embodiments, the fluid reservoir has a capacity from about 20 to about 100 milliliters. In some embodiments, the fluid reservoir has a capacity from about 30 to about 60 milliliters.

In one embodiment, there is disclosed a method for the treatment of one or more areas of acne vulgaris on a chest or a back of a patient comprising activating a spray activator; and spraying a quantity a liquid solution comprising an antibiotic of the lincomycin family on the one or more areas. In some embodiments, the quantity comprises from about 0.05 to about 0.5 milliliters for each activation of the spray activator. In some embodiments, the method also includes spraying at least twice daily. In some embodiments, the method also includes spraying at least once a day for a period of at least 2 weeks. In some embodiments, the liquid solution further comprises benzoyl peroxide. In some embodiments, the liquid solution has a pH of about 4 to about 6. In some embodiments, the liquid solution comprises about 1 to about 30 weight percent benzoyl peroxide and about 0.01 to about 5 weight percent clindamycin or a pharmaceutically acceptable salt or ester thereof.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the invention, including all features which would be treated as equivalents thereof by those skilled in the art to which this invention pertains.

1. A system comprising:
a fluid reservoir;
a liquid solution within the reservoir, the solution comprising an antibiotic of the lincomycin family;
a spray nozzle;
a spray activator; and
a tubular connection connecting the spray nozzle and the fluid reservoir.

2. The system of claim 1, wherein a quantity of the liquid solution is adapted to be transported from the fluid reservoir to the spray nozzle when the spray activator is engaged.

3. The system of claim 2, wherein the quantity comprises from about 0.05 to about 0.5 milliliters for each activation of the spray activator.

4. The system of claim 1, wherein the antibiotic comprises clindamycin phosphate or a pharmaceutically acceptable salt or ester thereof.

5. The system of claim 1, the liquid solution further comprising a peroxide.

6. The system of claim 5, wherein the peroxide comprises benzoyl peroxide.

7. The system of claim 1, wherein the liquid solution has a viscosity from about 0.01 to about 100 centipoise.

8. The system of claim 1, wherein the spray activator comprises a manual pump.

9. The system of claim 1, wherein the liquid solution has a viscosity from about 0.1 to about 10 centipoise.

10. The system of claim 1, wherein the fluid reservoir has a capacity from about 20 to about 100 milliliters.

11. The system of claim 1, wherein the fluid reservoir has a capacity from about 30 to about 60 milliliters.

12. A method for the treatment of one or more areas of acne vulgaris on a chest or a back of a patient comprising:
activating a spray activator; and
spraying a quantity a liquid solution comprising an antibiotic of the lincomycin family on the one or more areas.

13. The method of claim 12, wherein the quantity comprises from about 0.05 to about 0.5 milliliters for each activation of the spray activator.

14. The method of claim 12, further comprising spraying at least twice daily.

15. The method of claim 12, further comprising spraying at least once a day for a period of at least 2 weeks.

16. The method of claim 12, wherein the liquid solution further comprises benzoyl peroxide.

17. The method of claim 12, wherein the liquid solution has a pH of about 4 to about 6.

18. The method of claim 12, wherein the liquid solution comprises about 1 to about 30 weight percent benzoyl peroxide and about 0.01 to about 5 weight percent clindamycin or a pharmaceutically acceptable salt or ester thereof.
